(19) United States
(12) Patent Application Publication
(10) Pub. No.: US 2017/0304575 A1
(43) Pub. Date: Oct. 26, 2017

(54) NASAL HIGH FLOW THERAPY DEVICE AND METHOD

(71) Applicant: L'Air Liquide, Société Anonyme pour l'Etude et l'Exploitation des Procédés Georges Claude, Paris (FR)

(72) Inventor: Thierry BOULANGER, Swarthmore, PA (US)

(21) Appl. No.: 15/493,416

(22) Filed: Apr. 21, 2017

Related U.S. Application Data
(60) Provisional application No. 62/325,727, filed on Apr. 21, 2016.

(30) Foreign Application Priority Data
Aug. 9, 2016 (EP) 16183447.8

(51) Int. Cl. A61M 16/06; A61M 16/00

(52) U.S. Cl. CPC A61M 16/0666; A61M 16/0683; A61M 16/0057; A61M 2016/0027; A61M 2205/18; A61M 2205/502; A61M 2016/003

(57) ABSTRACT

The application describes a nasal sub-system (21) having:
a hollow body (22) with an inner chamber (22a) and an inlet (22b) for receiving a respiratory gas, and
a pair of nasal prongs (23, 24), in fluid communication with the inner chamber 22a of the hollow body 22, each nasal prong (23, 24) comprising a pair of inner channels (23b, 23c; 24b, 24c), each inner channels (23b, 23c; 24b, 24c) comprising a first channel (23b; 24b) and a second channel (23c; 24c) arranged in parallel, each first passage (23b; 24b) fluidly connecting the internal chamber (22a) of the hollow body (22) with a nostril (13, 14) of the patient (1), and each second passage (23c, 24c) fluidly connecting a nostril (13, 14) with a vent conduit (25) arranged in the hollow body (22) and in fluid communication with the atmosphere via at least one venting port (25a).

### CROSS REFERENCE TO RELATED APPLICATIONS

This application claims priority to European patent application no. EP 16183447.8 filed Aug. 9, 2016; and claims the benefit of priority under 35 U.S.C. §119 (a) and (b) to U.S. provisional patent application No. 62/325,727 filed Apr. 21, 2016, the entire contents of which are incorporated herein by reference.

### BACKGROUND

The invention concerns a nasal sub-system comprising two prongs that are inserted into the patient's nostrils for delivering a respiratory gas to a patient in need thereof, a gas delivery assembly including such a nasal sub-system and a measurement module, and a gas delivery apparatus comprising a high flow generator for delivering a respiratory gas to a patient by means of such a gas delivery assembly.

Nasal High Flow Therapy or NHFT is a widespread therapy used in many hospitals, especially in acute care services, against respiratory distresses. It does not require any mechanical ventilation and it can help some patients avoid being intubated.

Basically, NHFT consists in providing a humidified, high flow of respiratory gas to a patient through a nasal cannula. Gaseous flows typically range from 10 to 60 L/min for adults and 1 to 10 L/min for infants.

The nasal cannula comprises two prongs that are inserted into the patient's nostrils, although not totally occluding them. The prongs deliver the gaseous flow that the patient inhales and the space between the outer portion of the prongs and the nostrils helps the expired gaseous flow to be vented upon exhalation.

NHFT provides many advantages compared to continuous oxygen treatments or even to non-invasive ventilation, e.g.:
the gaseous flow can be enriched with Oxygen (O₂) up to 100%, i.e. the respiratory gas can be composed of air, O₂-enriched air (>22 vol % of O₂) or pure O₂;
the set flow can be greater than the inspiratory peak flow of the patient so as to limit his work of breathing, thereby assisting in the avoidance of fatigue of respiratory muscles.
the flow helps by flushing out CO₂ from the dead space of the upper airways, thereby enhancing the ventilation.
the flow and the space between the prongs and the nostrils create a positive pressure, which exhibits a beneficial action on ventilation.

On top of that, this therapy is extremely simple as:
only a flow and a level of oxygen have to be set by the physician or similar;
the nasal cannula is easy to be put in place; and
the patient can speak and even eat, while receiving the therapy.

However, today, medical service providers do not have easy access to parameters that might help them in setting the right gas flow and gas concentration for obtaining the best therapy for a given patient. Instead, they usually rely only on oxygen saturation (i.e. SpO₂).

For instance, in some cases, a physician may decide to increase the O₂ concentration for reaching an adequate O₂-saturation in the blood of the patient, whereas increasing the flow of gas would have been a better solution. In other instances, a physician may decide to provide a higher flow of gas, but such a high flow may produce significant pressure levels that can be detrimental over time for some patients. In that case, increasing the O₂ concentration in lieu of the gas flow might be a better solution.

Furthermore, it happens that some patients under NHFT persist in their respiratory distress and have to be intubated eventually, despite the NHFT treatment. One indicator of a possible future intubation is a persistent dyspnea despite the NHFT therapy or modification of the minute volume ventilation; however, this indicator is not easily assessed by medical service providers.

It is clear that giving access to parameters such as the intra-nasal pressure, minute ventilation and respiratory rate of patients under NHFT would be valuable for medical service providers.

### SUMMARY

It is one purpose of the present invention to provide additional parameters to medical service providers, helping them to optimize the treatment dosing and administration parameters and to improve the care delivered to the patient, potentially avoiding intubation or reducing the length of stay.

One solution according to the present invention concerns a gas delivery assembly comprising a nasal sub-system to be connected to a nose of a patient for delivering a respiratory gas to the patient, and a measurement module, wherein the nasal sub-system comprises:
a hollow body with an inner chamber and an inlet for receiving a respiratory gas, and
a pair of nasal prongs in fluid communication with the inner chamber of the hollow body, each nasal prong comprising a pair of inner channels, each inner channels comprising a first channel and a second channel arranged in parallel, each first passage fluidly connecting the internal chamber of the hollow body with a nostril of the patient, and each second passage fluidly connecting a nostril with a vent conduit arranged in the hollow body and in fluid communication with the atmosphere via at least one venting port, and wherein the measurement module comprises a main module body in fluid communication with the nasal sub-system, said main module body comprising:
a first inner gas conduct in fluid communication with the outlet port of the nasal sub-system, and
a hollow expansion portion comprising an inner volume in fluid communication with the vent conduit of the nasal sub-system.

Depending on the embodiment, a gas delivery assembly according to the present invention can comprise one or more of the following features:
the vent conduit of the hollow body of the nasal sub-system is in fluid communication with the internal chamber of the hollow body via each pair of inner channels.
an internal separation wall arranged in each prong of the nasal sub-system separates the first passage from the second passage of each prong.
the nasal prongs of the nasal sub-system are sized so as to match the inner walls of the patient's nostrils.
the prongs of the nasal sub-system have a conical or tronconical general shape.
a supplementary conduit is arranged in one of the two prongs of the nasal sub-system, said supplementary conduit comprising an inlet port located into one of the second conduits of the nasal prongs.
the inlet port of the supplementary conduit of the nasal sub-system is located in the vicinity of the free end of one of the nasal prongs.
a supplementary conduit of the nasal sub-system is arranged in one of the two prongs of the nasal sub-system, said supplementary conduit comprising an outlet port located into the vent conduit.
the supplementary conduit of the nasal sub-system comprises an outlet port located in the vicinity of the venting port of the vent conduit.
the nasal sub-system further comprises a fixing system for maintaining the nasal prongs in position into the patient's nostrils.
the first inner gas conduct of the main module body of the measurement module is further fluidly connected to a first port of the main module body, said first port comprising a first exit orifice for the gas.
the inner volume of the expansion portion of the main module body of the measurement module is in fluid communication with a second and a third ports of the main module body.
the main module body is attached to the nasal sub-system.

Another embodiment of the present invention concerns a gas delivery apparatus comprising a high flow generator for delivering a respiratory gas to a patient and a gas delivery assembly according to the present invention, especially as described hereabove.

Depending on the embodiment, a gas delivery apparatus according to the present invention can comprise one or more of the following features:
it further comprises a processing unit fluidly connected to the nasal sub-system.
it further comprises a processing unit electrically connected to the high flow generator.
it further comprises a processing unit comprising two pressure sensors electrically connected to a controller for providing pressure signals to said controller.
the processing unit further comprises a visual interface electrically connected to the controller for displaying data issued by the controller.
the processing unit further comprises a visual interface such as a screen or display.

### BRIEF DESCRIPTION OF THE DRAWINGS

Other features, aspects and advantages of the present invention will become apparent from the following detailed description when taken in conjunction with the accompanying drawings which illustrates, by way of examples, the present invention, among which:

FIG. 1 is a schematic representation of a gas delivery assembly comprising a nasal interface according to the prior art,

FIG. 2 is a schematic representation of the nasal interface of FIG. 1,

FIG. 3 represents an embodiment of a gas delivery assembly comprising a nasal interface according to the present invention,

FIG. 4 represents an embodiment of a nasal interface of a gas delivery assembly according to the present invention,

FIG. 5 represents a supplementary module of a gas delivery assembly that is directly attached to the nasal interface of FIG. 4,

FIG. 6 represents an embodiment of a processing unit of the gas delivery assembly of FIG. 3, and

FIG. 7 represents a supplementary module of a gas delivery assembly that is remotely connected to the nasal interface of FIG. 4.

### DESCRIPTION OF PREFERRED EMBODIMENTS

FIG. 1 shows a gas delivery equipment comprising a cannula assembly 2 according to the prior art that fluidly connects a patient 1 to a high flow generator 3, such as the device called Precision FIow™ distributed by Vapotherm or the device called Optiflow™ distributed by Fisher & Paykel.

The cannula assembly 2 comprises an elongated flexible tubing 20, such as a flexible hose, comprising, at one end, a nasal sub-system 21 connected to the nose of patient 1 thereby delivering respiratory gas to the patient 1. The elongated flexible tubing 20 is connected, by its other end, to the high flow generator 3.

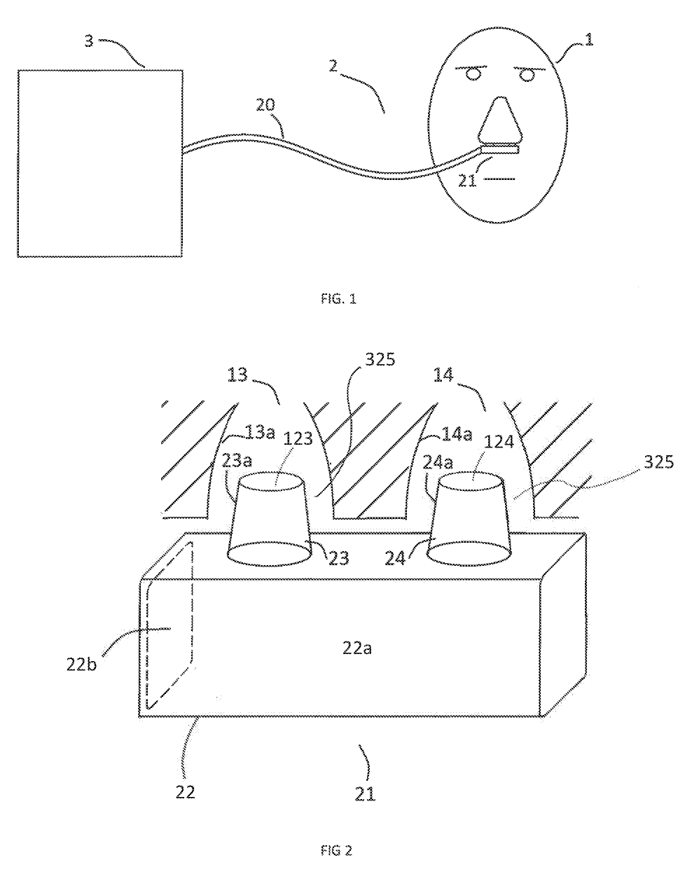

As detailed in FIG. 2, the nasal sub-system 21 comprises a hollow body 22 with an inner chamber 22a, acting as a manifold for receiving the respiratory gas conveyed and fed by the flexible tubing 20, and nasal prongs 23, 24, i.e. little nozzles, in fluid communication with the lumen of the inner chamber 22a of the hollow body 22, that are inserted, in use, into the nostrils of the patient 1 for delivering a respiratory gas to the patient 1 in need thereof.

Each nasal prong 23, 24 comprises a unique inner channel or passage 123, 124 for conveying the gas from the inner chamber 22a of the hollow body 22 to the nostrils 13, 14 of the patient 1.

The hollow body 22 can have various shapes, for example be of circular or rectangular cross section. The gas fed by the tubing 20 travels successively in the chamber 22a of the hollow body 22 and then in each nasal prongs 23, 24, and eventually is distributed into the nostrils 13, 14 of the patient.

With such a prior art equipment, a gas tight connection/insertion of the prongs 23, 24 into the nostrils 13, 14 is not ensured as the outer peripheral walls 23a, 24a of the nasal prongs 23, 24, respectively, are not operated in a manner that provides a sealed contact with the inner walls 13a, 14a of the nostrils 13, 14, respectively. This means that a spacing 325 always exists between the inner walls 13a, 14a of the nostrils 13, 14, and the outer peripheral walls 23a, 24a of the nasal prongs 23, 24, respectively, leading to gas loses.

The gaseous flow delivered by the high flow generator 3 enters into the inner chamber 22a of the hollow body 22 and is then directed to the prongs 23, 24 so that upon inhalation, only a part of the gaseous flow is inspired by the patient 1, as the rest is unfortunately vented to the atmosphere, while escaping through the spacing 325 that inevitably exists.

In opposite, upon expiration, both gaseous flows coming, on the one hand, from the high flow generator 3 and, on the other hand, exhaled by the patient do circulate in said spacing 325, thereby creating a positive expiratory pressure before being vented to the atmosphere.

Such architecture is not ideal as it prevents the use of gas mixtures including noble gases such as Helium due to cost issues due to significant losses of Helium or other medicinal gaseous or vaporous contents in the gas feed.

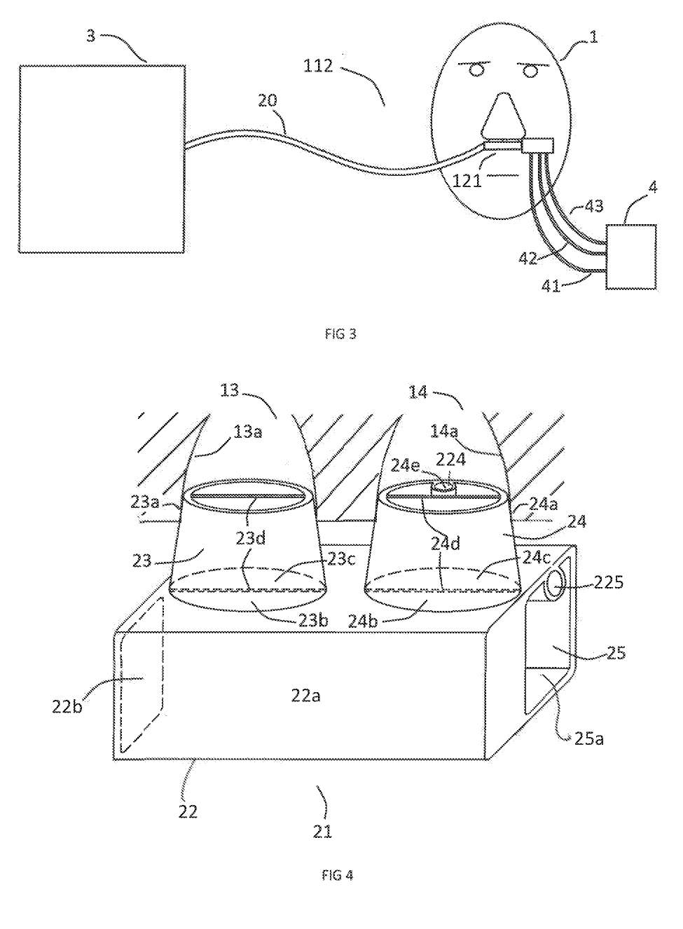

FIG. 3 represents a first embodiment of a gas delivery assembly comprising a first embodiment of a cannula assembly 112 according to the present invention for fluidly connecting a patient 1 to a high flow generator 3 as in FIG. 1.

As in FIGS. 1 and 2, the cannula assembly 112 comprises an elongated flexible tubing 20 connected, on the one hand, to the high flow generator 3, and, on the other hand, to a nasal sub-system 121 inserted into the nose of patient 1, thereby delivering respiratory gas to the patient 1.

According to the present invention, the nasal sub-system 121 comprises further measurement devices for measuring and transmitting physiological signals, such as gas pressure and/or flowrate, to a processing unit 4. These additional measurement devices comprise three measurement lines, namely flexible tubings 41, 42, 43 that are fluidly connected to the nasal sub-system 121 as shown in FIG. 3.

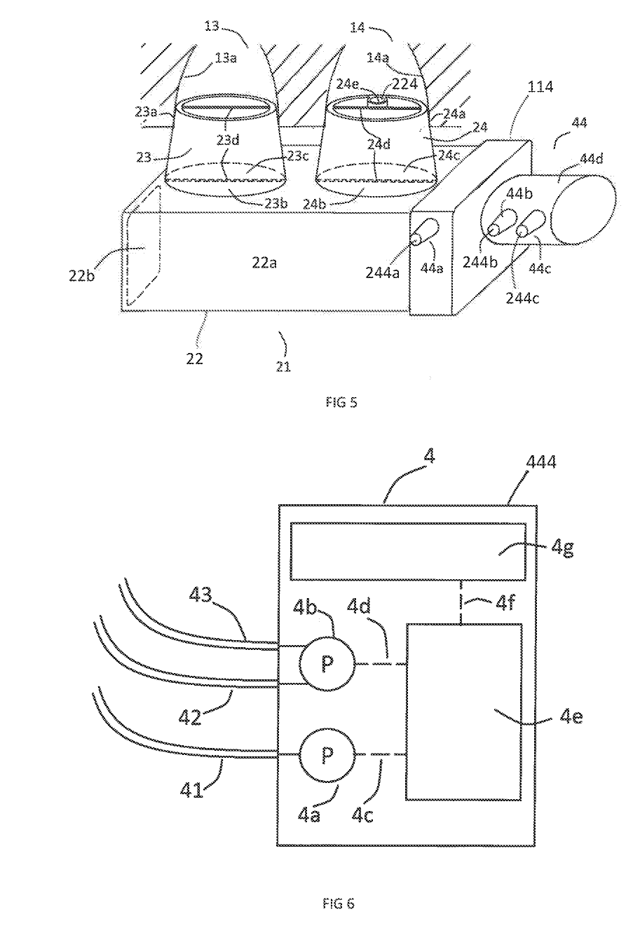

FIG. 4 shows an embodiment of a nasal sub-system 21 according to the present invention. It comprises, as in FIG. 2, a hollow body 22 with an inner chamber 22a and a gas inlet 22b for receiving a respiratory gas fed by the flexible tubing 20 and a pair of prongs 23, 24 in fluid communication with the inner chamber 22a of the hollow body 22 for delivering gas to the patient's nostrils 13, 14.

Here also, the hollow body 22 can have any suitable shape, for example have a circular or rectangular section.

In this embodiment, the nasal prongs 23, 24 that are inserted in the patient's nostrils 13, 14 have been designed, i.e. sized and/or dimensioned, so as to exhibit a larger diameter and hence better match the inner walls 13a, 14a of the patient's nostrils 13, 14, thereby removing or minimizing the spacing 325 between the peripheral walls 23a, 24a of the prongs 23, 24 and the inner walls 13a, 14a of the nostrils 13, 14. With such enlarged prongs 23, 24, the gas tightness is improved compared to the embodiment of FIG. 2 as the peripheral walls 23a, 24a of the prongs 23, 24 are in contact with the inner walls 13a, 14a of the nostrils 13, 14, i.e. as no or almost no spacing 325 exists anymore in-between. Preferably, the prongs 23, 24 have a conical or tronconical general shape as shown in FIG. 4 so as to better fit the inner shape of the nostrils 13, 14 and further to facilitate the insertion of the prongs 23, 24 into said nostrils 13, 14.

In the embodiment of FIG. 4, the nasal prongs 23, 24 comprise each two inner channels or passages 23b, 23c, 24b, 24c arranged in parallel for directing the gas flows during inspiration and expiration phases.

In each prong 23, 24, a first passage 23b, 24b fluidly connects the internal chamber 22a of the hollow body 22 with a nostril 13, 14 of the patient 1, whereas a second passage 23c, 24c fluidly connects the nostril 13, 14 with a vent conduit 25 arranged in the hollow body 22, which is in fluid communication with the atmosphere, via one or several venting port 25a. The vent conduit 25 does not communicate directly with the internal chamber 22a of the hollow body 22.

The first passage 23b, 24b and the second passage 23c, 24c of each prong 23, 24 is obtained in vertically separating each nasal prong, i.e. each little nozzle, by an internal separation wall 23d, 24d, i.e. an inner thin wall separates each prong in two parts in the vertical way as shown in FIG. 4.

With such a configuration, the gaseous flow, such as air, conveyed by the tubing 20 enters into the internal chamber 22a of the hollow body 22, travels through the first channels 23b, 24b and is then delivered by the pair of prongs 23, 24 to the nostrils 13, 14 of the patient 1.

During the inhalation phases of the patient 1, a part of the gaseous flow is inspired by the patient 1, whereas the rest of the gaseous flow is directed to the second channels 23c, 24c, which are fluidly connected to the vent conduit 25 thereby allowing this excess of gas escaping to the atmosphere by venting port(s) 25a.

In contrast, during the exhalation phases of the patient 1, both gaseous flows coming from the internal chamber 22a of the hollow body 22, on the one hand, and exhaled by the patient, on the other hand, pass through the second channels 23c, 24c and are vented to the atmosphere thanks to the vent conduit 25 and venting port 25a, thereby creating a positive expiratory pressure or PEP.

Further, in one of the prongs 23, 24, a supplementary conduit 24e is arranged. Said supplementary conduit 24e comprises an inlet port 224 port located into a second conduit 23c, 24c, preferably in the vicinity of the free end of the nasal prong 23 in which the supplementary conduit 24e is arranged, and an outlet port 225 located into the vent conduit 25, preferably in the vicinity of the venting port 25a. This supplementary conduit 24e allows collecting some important physiological parameters, such as the gas pressure in the patient's nostril 14 as explained below in connection with FIG. 5.

The nasal sub-system 21 can be made of silicon material so as to be light for the patient 1. Further, it preferably comprises one or several straps, a headgear or similar a fixing system (not shown) for maintaining the nasal sub-system 21 in position in the nostrils 12, 14 of the patient 1.

FIG. 5 shows the nasal sub-system 21 of FIG. 4 to which an additional measurement module 44 has been attached, which can be used for collecting information or data related to the gas flows to and/or from the patient, which can be useful to the medical team.

More precisely, the measurement module 44 comprises a main module body 144 with fixation elements (not shown) for fixing or attaching the measurement module 44 to the nasal sub-system 21. The main module body 144 comprises a first inner gas conduct fluidly linking or connecting the outlet port 225 to a first port 44a comprising a first exit orifice 244a through which the gas can exit the first inner gas conduct and first port 44a or connection. The first port 44a is itself fluidly connected to a first flexible tubing 41 for feeding the first flexible tubing 41 with gas.

In the embodiment of FIG. 5, the main module body 144 further comprises an expansion portion 44d, such as a cylindrical part, that is hollow. The lumen or inner volume of the expansion portion 44d is in fluid communication, on the one end, with the vent conduit 25, through the outlet port 25a, and on the other hand, with a second and a third ports 44b, 44c or connections, that are connected to second and third flexible tubings 42, 43.

The second and third ports 44b, 44c comprise second and third exit orifices 244b, 244c, respectively, for delivering pressurized gas to the second and third flexible tubings 42, 43, respectively.

The second and third flexible tubings 42, 43 are themselves connected to the processing unit 4, such as a microcontroller or microprocessor.

The gas travelling into the lumen of the expansion portion 44d is submitted to a slight pressure drop during its propagation towards the second and third ports 44b, 44c. This structure constitutes a flow sensor. Gas pressure measurements can be made through the second and third ports 44b, 44c and then transmitted to the processing unit 4 by the second and third flexible tubings 42, 43 where that can be processed.

Gas pressure measurements made by the module 44 include the instantaneous gas pressure measured in the nostril 14 of the patient 1 as well as the gas pressure before and after any pressure drop into the lumen of the expansion portion 44d.

Once processed, those data constitute valuable information to the medical team.

FIG. 6 represents an embodiment of a processing unit 4 that can be fluidly connected to the nasal sub-system 21 according to the present invention as shown in FIG. 5.

The processing unit 4 comprises a casing 444 that embeds two pressure sensors 4a, 4b.

First sensor 4a is connected to the first tubing 41 connected to the first port 44a for thereby monitoring the instantaneous pressure in one of the patient's nostrils 13, 14. First pressure sensor 4a is electrically connected with cable 4c to a controller 4e which will further transform and process the pressure information. For instance, the average pressure over a given period of time, for example over 10 sec, will help the medical service provider in adjusting the gaseous flow delivered by the High Flow Generator 3 in order to keep the pressure at a safe level in the patient's upper airways.

Controller 4e can comprise a microprocessor running one or several algorithms.

Useful information may result from the analysis by controller 4e of the instantaneous pressure provided by the first pressure sensor 4a. Indeed, upon inhalation, the instantaneous pressure will drop below the average pressure as most of the flow will be inspired by the patient 1. On the contrary, a slight overpressure will happen upon exhalation of gas by the patient 1 as the total flow will be the sum of the flow coming from the High Flow Generator 3 and the flow exhaled by the patient 1.

Hence, providing controller 4e with processing means to process and analyze these pressure swings around the mean pressure value will give access to the patient's respiratory breath, which is also a very valuable indicator for the medical service provider or other members of the medical team.

In the same spirit, the second pressure sensor 4b is connected to the second and third tubings 42, 43 which are respectively connected to the second and third ports 44b, 44c. The second pressure sensor 4b is preferably a differential pressure sensor allowing measuring the slight pressure difference, i.e. pressure drop, that exists between the second and third tubing 42, 43. The second sensor 4b is electrically connected with cable 4d to controller 4e which will further transform and process the pressure information delivered by the second sensor 4b.

For instance, based on the geometry of the expansion portion 44d, controller 4e is able to determine the absolute value of the flow, relying on a stored lookup table, for translating the differential pressure readings from pressure sensor 4b to actual gas flow values.

Having a measure of the instantaneous flow, controller 4e can calculate an average flow over a given period of time, for example over the course of a breath, which will give a good approximation of the continuous flow generated by the High Flow generator 3.

In order to have all these parameters (such as pressure, respiratory rate, inspiratory volumes and minute volume) accessible to the physician or the medical team, controller 4e can electronically transfer these information by means of an electric cable 4f or other data transmission systems (e.g. wireless radio or infrared communication) to a visual interface 4g or similar data display system.

The processing unit 4 can further embed additional signal systems, such as an acoustic alarm, for alerting the medical team or the user in cases where the processing unit 4 detects that one parameter is out of a given range of threshold values.

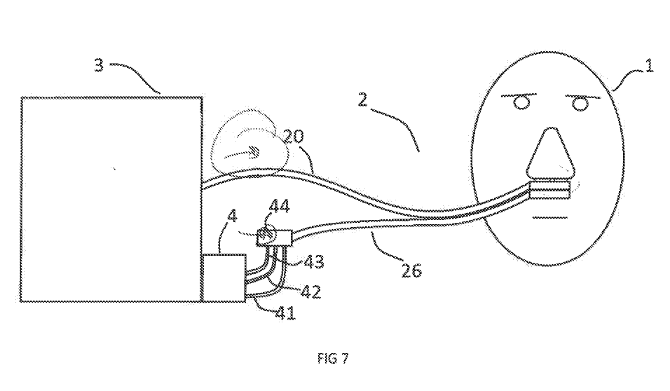

Furthermore, the processing unit 4 can be an independent device (cf. FIG. 4) or can be integrated into or attached to the High Flow generator 3 (cf. FIG. 7).

FIG. 7 shows another embodiment of the nasal sub-system 21 of FIG. 4 fluidly connected to an additional measurement module 44 similar to the one of FIG. 5 for collecting information or data related to the gas flows to and/or from the patient.

However, in this embodiment, the additional measurement module 44 is not directly attached to the nasal sub-system 21 of FIG. 4 but is connected to it by means of one or several conducts 26, such as a long, flexible hose.

In other words, the additional measurement module 44 is remote from the patient and located close to a processing unit 4, as the one of FIG. 6, to which it is fluidly connected by means of the first, second and third tubings 41, 42, 43 as explained above. Of course, the additional measurement module 44 could be also directly embedded in the processing unit 4 thereby limiting the number of tubings, hoses or similar.

The processing unit 4 can be further attached or plugged to the ventilator 3 as shown in FIG. 7 or constitute an independent device electrically linked to the ventilator 3.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A gas delivery assembly comprising a nasal sub-system (21) to be connected to a nose of a patient for delivering a respiratory gas to the patient, and a measurement module (44), wherein the nasal sub-system (21), comprises:
a hollow body (22) with an inner chamber (22a) and an inlet (22b) for receiving a respiratory gas, and
a pair of nasal prongs (23, 24), in fluid communication with the inner chamber 22a of the hollow body 22, each nasal prong (23, 24) comprising a pair of inner channels (23b, 23c; 24b, 24c), each inner channels (23b, 23c; 24b, 24c) comprising a first channel (23b; 24b) and a second channel (23c; 24c) arranged in parallel, each first passage (23b; 24b) fluidly connecting the internal chamber (22a) of the hollow body (22) with a nostril (13, 14) of the patient (1), and each second passage (23c, 24c) fluidly connecting a nostril (13, 14) with a vent conduit (25) arranged in the hollow body (22) and in fluid communication with the atmosphere via at least one venting port (25a), and
wherein the measurement module (44) comprises a main module body (144) in fluid communication with the nasal sub-system (21), said main module body (144) comprising:
a first inner gas conduct in fluid communication with an outlet port (225) of the nasal sub-system (21), and
a hollow expansion portion (44d) comprising an inner volume in fluid communication with the vent conduit (25) of the nasal sub-system (21).

2. The gas delivery assembly according to claim 1, wherein the vent conduit (25) of the hollow body (22) of the nasal sub-system (21) is in fluid communication with the internal chamber (22a) of the hollow body (22) via each pair of inner channels (23b, 23c; 24b, 24c).

3. The gas delivery assembly according to claim 1, wherein an internal separation wall (23d, 24d) arranged in each prong (23, 24) of the nasal sub-system (21) separates the first passage (23b, 24b) from the second passage (23c, 24c) of each prong (23, 24).

4. The gas delivery assembly according to claim 1, wherein the nasal prongs (23, 24) of the nasal sub-system (21) are sized so as to match the inner walls (13a, 14a) of the patient's nostrils (13, 14).

5. The gas delivery assembly according to claim 1, wherein the prongs (23, 24) of the nasal sub-system (21) have a conical or tronconical general shape.

6. The gas delivery assembly according to claim 1, wherein a supplementary conduit (24e) is arranged in one of the two prongs (23, 24) of the nasal sub-system (21), said supplementary conduit (24e) comprising an inlet port (224) port located within one of the second conduits (23c, 24c) of the nasal prongs (23, 24).

7. The gas delivery assembly according to claim 6, wherein the inlet port (224) of the supplementary conduit (24e) of the nasal sub-system (21) is located in a vicinity of a free end of one of the nasal prongs (23, 24).

8. The gas delivery assembly according to claim 1, wherein a supplementary conduit (24e) of the nasal sub-system (21) is arranged in one of the two prongs (23, 24) of the nasal sub-system (21), said supplementary conduit (24e) comprising an outlet port (225) located into the vent conduit (25).

9. The gas delivery assembly according to claim 8, wherein the supplementary conduit (24e) of the nasal sub-system (21) comprises an outlet port (225) located in a vicinity of the least one venting port (25a) of the vent conduit (25).

10. The gas delivery assembly according to claim 1, wherein the nasal sub-system (21) further comprises a fixing system for maintaining the nasal prongs (23, 24) in position into the patient's nostrils (13, 14).

11. The gas delivery assembly according to claim 1, wherein the first inner gas conduct of the main module body (144) of the measurement module (44) is further fluidly connected to a first port (44a) of the main module body (144), said first port (44a) comprising a first exit orifice (244a) for a first gas flow from the measurement module (44).

12. The gas delivery assembly according to claim 1, wherein an inner volume of an expansion portion (44d) of the main module body (144) of the measurement module (44) is in fluid communication with a second and a third port (44b, 44c) of the main module body (144).

13. The gas delivery assembly according to claim 1, wherein the main module body (144) is attached to the nasal sub-system (21).

14. The gas delivery apparatus and a gas delivery assembly according to claim 1 further comprising a high flow generator (3) fluidly connected to the gas delivery assembly for delivering a respiratory gas to a patient.

15. The gas delivery apparatus according to claim 14, further comprising a processing unit (4) fluidly connected to the nasal sub-system (21).

16. The gas delivery apparatus according to claim 14, further comprising a processing unit (4) electrically connected to the high flow generator (3).

17. The gas delivery apparatus according to claim 14, further comprising a processing unit (4) comprising two pressure sensors (4a, 4b) electrically connected to a controller (4e) for providing pressure signals to said controller (4e).

18. The gas delivery apparatus according to claim 17, wherein the processing unit (4) further comprises a visual interface (4g) electrically connected to the controller (4e) for displaying data issued by the controller (4e).
